(19) United States
(12) Patent Application Publication
(10) Pub. No.: US 2025/0339853 A1
(43) Pub. Date: Nov. 6, 2025

(54) MULTI-CHAMBER PIPETTE TIP ASSEMBLY FOR AUTOMATED SAMPLE PREPARATION AND DIAGNOSTIC TESTING

(71) Applicant: IDEXX Laboratories, Inc., Westbrook, ME (US)

(72) Inventor: Chase Leffers, Boston, MA (US)

(73) Assignee: IDEXX Laboratories, Inc., Westbrook, ME (US)

(21) Appl. No.: 19/194,820

(22) Filed: Apr. 30, 2025

Related U.S. Application Data
(60) Provisional application No. 63/642,037, filed on May 3, 2024.

(51) Int. Cl. B01L 3/02

(52) U.S. Cl. CPC B01L 3/0237; B01L 2200/0621; B01L 2300/027; B01L 2300/0867; B01L 2400/0478; B01L 2400/086

(57) ABSTRACT

A tip assembly comprising a body defining a first end, a second end opposite the first end, and a plurality of chambers therein, the plurality of chambers comprising: an intake chamber defining an inlet a the second end for passing a biological sample and comprising an intake piston positioned at least partially in the intake chamber; an output chamber defining an outlet at the second end for passing the biological sample and comprising an output piston positioned at least partially in the chamber; a mixing chamber positioned between the intake chamber and the output chamber and comprising a mixing piston positioned at least partially in the mixing chamber; a primary channel extending between the intake chamber and the mixing chamber; and a secondary channel extending between the mixing chamber and the output chamber.

### CROSS-REFERENCE TO RELATED APPLICATION

This application claims the benefit of co-pending U.S. Provisional Patent Application No. 63/642,037, filed May 3, 2024 for “Multi-Chamber Pipette Tip Assembly For Automated Sample Preparation And Diagnostic Testing,” which is hereby incorporated by reference in its entirety including the drawings.

### TECHNICAL FIELD

The present specification relates to sample preparation devices, and more particularly, to consumable sample preparation systems and methods for preparing biological samples.

### BACKGROUND

To perform antigen testing on fecal samples, ear swabs, or other biological samples, a user may collect a biological sample for immediate testing or for shipment to a testing facility or may perform the tests in-clinic. This requires either the user or the testing facility to handle the biological sample, which may result in contamination, sample spoilage, or incorrect preparation. In addition, traditional sample collection systems and methods often involve multiple stages and can require the use of multiple consumable products. Multi-step preparation systems and methods can take excessive amounts of time to prepare each biological sample for testing. Furthermore, multi-step sample preparation methods require the use of multiple consumable products, which can increase costs and increase the likelihood of contamination during sample transfer.

Accordingly, a need exists for alternative sample collection apparatuses for collecting biological samples with a simple to use, compact tool for performing automated sample preparation in a single consumable product.

### SUMMARY

In one embodiment, the tip assembly includes a body having a first end, a second end opposite the first end and defines a plurality of chambers therein. The plurality of chambers includes an intake chamber defining an inlet for passing a biological sample and having an intake piston positioned at least partially in the intake chamber. The tip assembly includes a mixer positioned at least partially within the intake chamber and coupled to the intake piston. The tip assembly also includes a first side chamber positioned in communication with the intake chamber and having a first side piston positioned at least partially within the first side chamber and a second side chamber in communication with the intake chamber having a second side piston positioned at least partially within the second side chamber. The tip assembly includes a primary channel extending between the first side chamber and the intake chamber and a secondary channel extending between the second side chamber and the intake chamber.

In another embodiment, a method for using a tip assembly includes loading a biological sample into an intake chamber through an inlet; moving an intake piston to rotate a mixer operatively coupled to the intake piston within the intake chamber; moving a first side piston to increase a first local pressure in a first side chamber such that a first fluid medium contained within the first side chamber is directed through a primary channel into the intake chamber to form a mixture of the biological sample and the first fluid medium; moving a second side piston to increase a second local pressure in a second side chamber such that a second fluid medium contained within the second side chamber is directed through a secondary channel into the intake chamber to add the second fluid medium to the mixture; and moving the intake piston to direct the mixture to exit the intake chamber through the inlet.

In another embodiment, a tip assembly including a body defining a first end, a second end opposite the first end, and a plurality of chambers therein. The plurality of chambers includes an intake chamber defining an inlet at the second end for passing a biological sample and comprising an intake piston positioned at least partially in the intake chamber; an output chamber defining an outlet at the second end for passing the biological sample and includes an output piston positioned at least partially in the chamber. The tip assembly includes a mixing chamber positioned between the intake chamber and the output chamber having a mixing piston positioned at least partially in the mixing chamber. The tip assembly includes a primary channel extending between the intake chamber and the mixing chamber and a secondary channel extending between the mixing chamber and the output chamber.

In another embodiment, a method for using a tip assembly, the method including moving an intake piston to draw a sample collection member and a collected biological sample into an intake chamber; moving a mixing piston to increase a pressure in a mixing chamber such that a fluid medium within the mixing chamber is directed through a primary channel into the intake chamber to form a mixture of the biological sample and the fluid medium; moving the mixing piston and moving the intake piston to create a pressure differential between the intake chamber and the mixing chamber such that the mixture is directed through the primary channel into the mixing chamber; moving an output piston and moving the mixing piston to create a pressure differential between the mixing chamber and an output chamber such that the mixture is directed through a secondary channel into the output chamber; and moving the output piston to direct at least a portion of the mixture to exit the output chamber through an outlet.

Additional features and advantages of the technology described in this disclosure will be set forth in the detailed description which follows, and in part will be readily apparent to those skilled in the art from the description or recognized by practicing the technology as described in this disclosure, including the detailed description which follows, the claims, as well as the appended drawings.

### BRIEF DESCRIPTION OF THE DRAWINGS

The embodiments set forth in the drawings are illustrative and exemplary in nature and not intended to limit the disclosure. The following detailed description of the illustrative embodiments can be understood when read in conjunction with the following drawings, where like structure is indicated with like reference numerals and in which:

FIG. 1 schematically depicts a perspective view of an example pipette platform having a pipette controller coupled to an example tip assembly via an adapter, according to one or more embodiments shown and described herein;

FIG. 2 schematically depicts a partial perspective view of the example pipette platform including a pipette controller, the adapter, and the tip assembly of FIG. 1, according to one or more embodiments shown and described herein;

FIG. 3 schematically depicts a cross-sectional view of the tip assembly and the adapter, according to one or more embodiments shown and described herein;

FIG. 4 schematically depicts a perspective view of the tip assembly, according to one or more embodiments shown and described herein;

FIG. 5 schematically depicts a cross-sectional view of the tip assembly of FIG. 4 including a fecal collection tip, according to one or more embodiments shown and described herein;

FIG. 6 schematically depicts a partial perspective view of the tip assembly of FIG. 4 including an ear swab tip, according to an alternative embodiment shown and described herein;

FIG. 7 schematically depicts a cross-sectional view of a tip assembly, according to one or more embodiments shown and described herein.

The present disclosure may be embodied in several forms without departing from its spirit or essential characteristics. The scope of the present disclosure is defined in the appended claims, rather than in the specific description preceding them. All embodiments that fall within the meaning and range of equivalency of the claims are therefore intended to be embraced by the claims.

### DETAILED DESCRIPTION

In modern medical and veterinary practices, biological sample testing is performed to diagnose biological maladies, detect antigens, and prepare treatment plans. To perform testing an analysis on biological samples, the samples must first be prepared. Typically, sample preparation of biological materials can include multiple steps. For instance, biological samples may need to be ground, dispersed in a solvent, mixed, prepared with a reagent, or the like prior to testing. Traditionally, each operation in the sample preparation process is performed in a separate consumable device. Using multiple consumable devices to prepare a single sample necessitates multiple transfer steps that can be time consuming and can increase the risk of contamination. Additionally, the use of multiple consumable devices can increase cost. Furthermore, relying on a user to manually transfer the biological sample and precisely measure amounts of solvents, reagents, dyes, or the like can lead to user error and potentially diminish the accuracy of test results.

To decrease preparation times, diminish costs, and increase the reliability of sample preparation, it is desirable to provide a handheld pipette platform that enables the automated sample preparation of biological matter within a multi-chamber consumable apparatus. A multi-chambered consumable tip can eliminate the need for intermediate preparation steps, thereby reducing the amount of consumable products used in the sample preparation process and minimizing costs. Furthermore, the multi-chambered consumable tip can increase the accuracy and repeatability of sample preparation steps by providing an automated preparation assembly that can collect, mix, aerate, dilute, filter, separate, and test biological samples all within a single consumable apparatus.

Directional terms as used herein—for example up, down, right, left, front, back, top, bottom—are made only with reference to the figures as drawn and are not intended to imply absolute orientation.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, it is in no way intended that an order or orientation be inferred. This holds for any possible non-express basis for interpretation, for example and without limitation, matters of logic with respect to arrangement of steps, operational flow, order of components, or orientation of components; plain meaning derived from grammatical organization or punctuation, and; the number or type of embodiments described in the specification.

As used herein, the singular forms “a,” “an” and “the” include one or more referents unless the context clearly dictates otherwise. Thus, for example, reference to “a” component includes aspects having a single component or multiple components, unless the context clearly indicates otherwise.

As used herein, the terms “first,” “second,” and “third” may be used interchangeably to distinguish one component from another and are not intended to signify location or importance of the individual components.

Approximating language, as used herein throughout the specification and claims, is applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value, or the precision of the methods or machines for constructing or manufacturing the components and/or systems. For example, the approximating language may refer to being within a 10 percent margin.

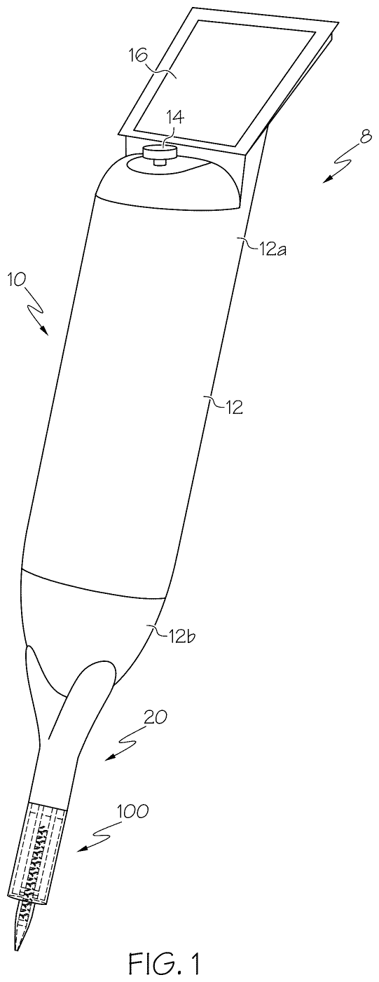

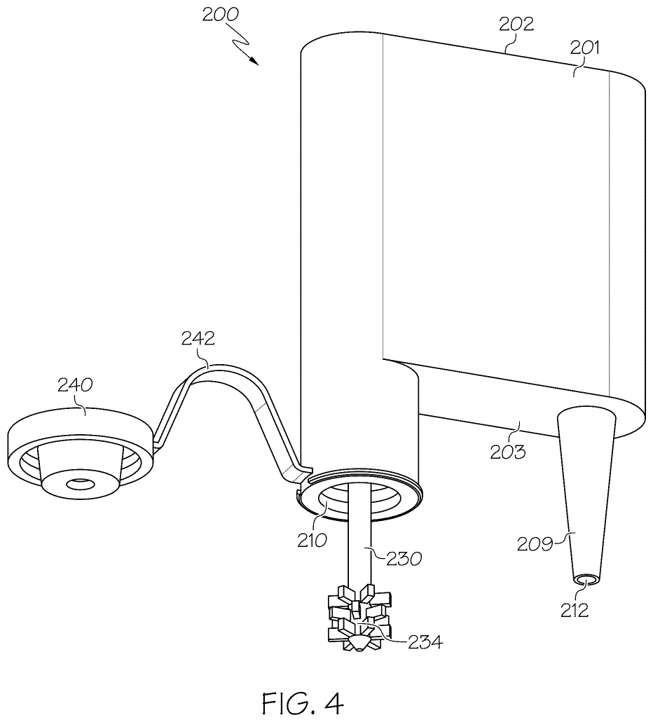

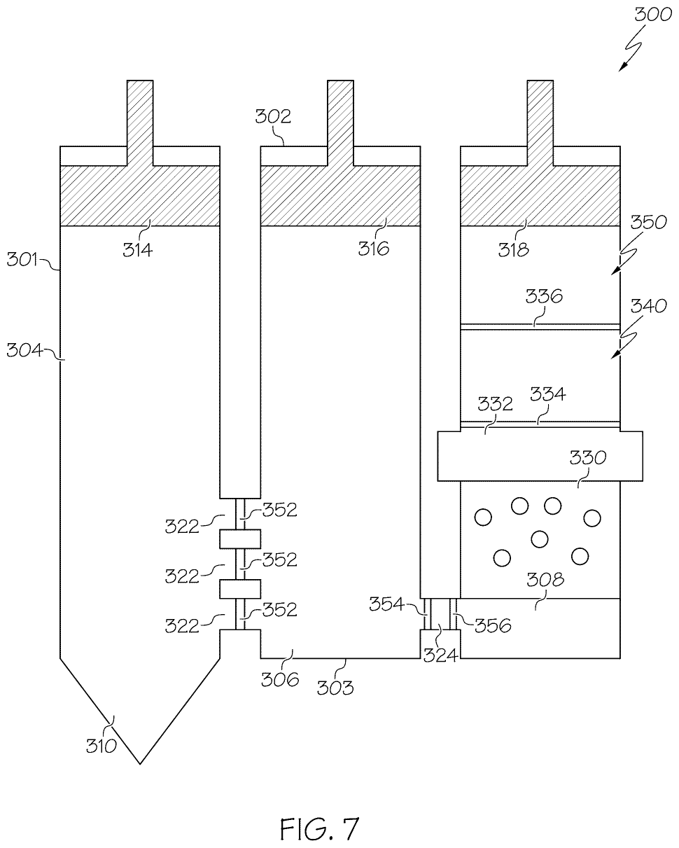

Referring now to FIG. 1, depicted is an illustrative embodiment of a pipette platform 8 including a pipette controller 10, an adapter 20, and a tip assembly 100 for automated sample preparation in a medical or veterinary setting. In some embodiments, the pipette controller 10 and the adapter 20 may form an integrated instrument while the tip assembly 100 may be a consumable component that can be coupled to the pipette controller 10 and/or the adapter 20 prior to each sample collection operation. The pipette platform 8 may accomplish one or more functions including, but not limited to, performing onboard mixing, filtering of a biological sample to segregate particular media, providing additional fluids to further process the biological sample, and/or test of the biological sample. The pipette controller 10 and adapter 20 are capable of receiving one or more configurations of attachments. The adapter 20 may receive the tip assembly 100, a tip assembly 200 (FIG. 4), and/or a tip assembly 300 (FIG. 7). The pipette controller 10 can accept customized tip embodiments, which each serve a specific purpose. In embodiments, a single adapter 20 may receive any one of the tip assembly 100, the tip assembly 200 (FIG. 4), or the tip assembly 300 (FIG. 7). Yet, in other embodiments, separate adapters 20 are utilized to receive a respective one of the tip assembly 100, the tip assembly 200 (FIG. 4), and the tip assembly 300 (FIG. 7).

In embodiments, the pipette controller 10 includes a shell 12, at least one user input 14, and a user interface 16. The shell 12 may include a handle end 12a and an opposite tip end 12b. The user input 14 and the user interface 16 provided at the handle end 12a with the adapter 20 provided at the tip end 12b. However, it should be understood that the user input 14 and the user interface 16 may be located at any suitable location of the shell 12. In embodiments, the user input 14 may be a button, switch, joystick, or the like to enable a user to control a function of the pipette controller 10. In embodiments, the user interface 16 may be a dial, screen, display or the like to present information to the user. The pipette controller 10 is comprises one or more actuators positioned within the shell 12. In embodiments, the actuators can each power a leadscrew to move a linear rider within an interior shaft positioned within the shell 12. The actuator can drive a lead screw or any other suitable structure to move within the one or more shafts positioned within the shell 12 of the pipette controller 10. In embodiments, the actuator may be a stepper motor or any other electronic, hydraulic, pneumatic, or other system capable of powering the lead screw. The lead screw may be coupled with a linear rider corresponding to the shape of the internal shaft to prevent the rider from rotating and force it to move up and down within the shaft while maintaining a seal against the interior walls of the shaft. The linear rider may be coupled with an O-ring to further ensure the seal of the shaft to prevent air from escaping and to prevent lubricants from progressing through the shaft toward the adapter 20. During operation of the actuator, the linear rider moves within the shaft to increase or decrease the volume of the shaft thereby compressing or expanding the air within the shaft to create a pneumatic force. In embodiments, the pipette controller 10 includes two or more internal shafts each coupled to an actuator to control the movement of each shaft's respective linear rider. Each shaft can operate independently of the one or more other shafts so that the pipette controller 10 may selectively control each actuator to selectively control the linear rider within each shaft.

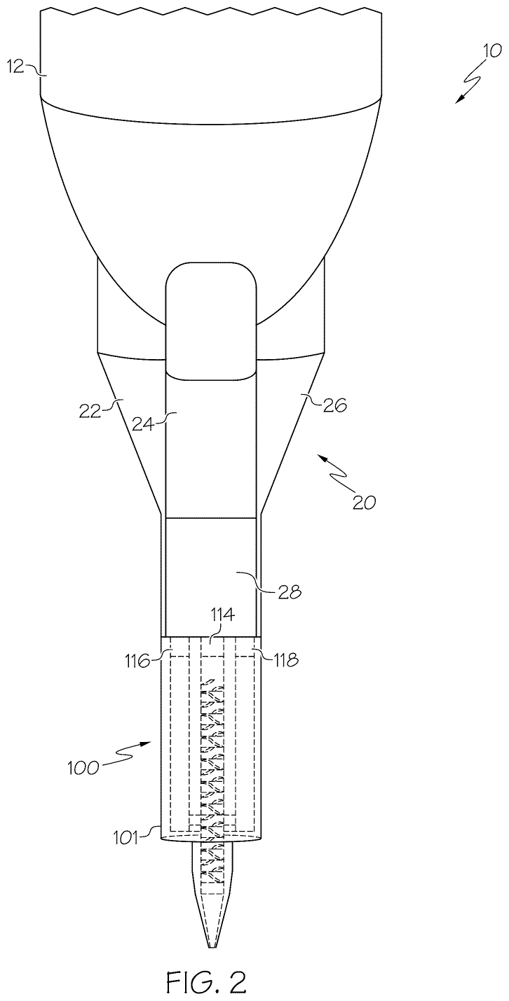

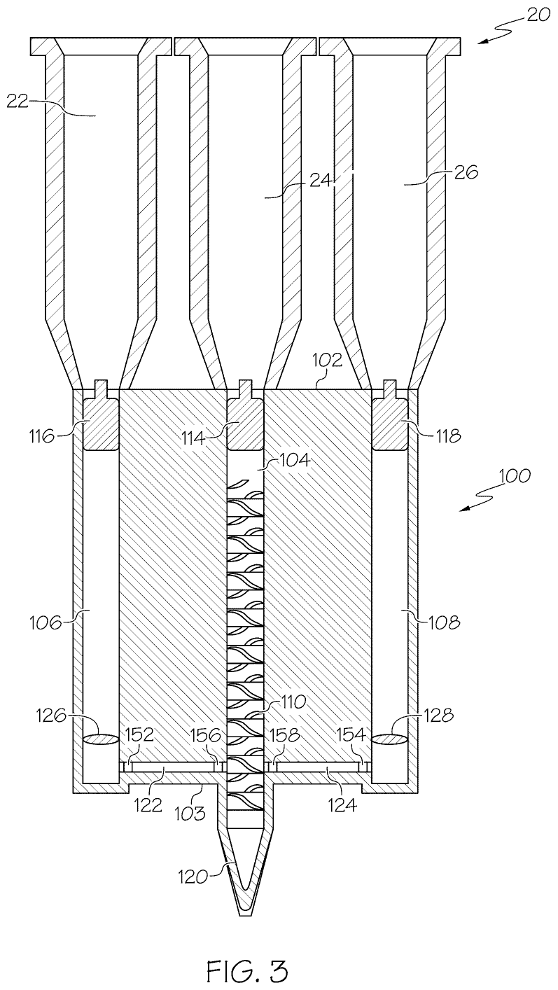

Referring now to FIGS. 2 and 3, the pipette platform 8 may be customizable by facilitating the attachment of the adapter 20 that serves to connect the one or more shafts within the shell 12 of the pipette controller 10 to the tip assembly 100, the tip assembly 200 (FIG. 4), or the tip assembly 300 (FIG. 7). The adapter 20 can have any suitable shape to engage each shaft of the pipette controller 10 with a corresponding chamber 104, 106, 108 (FIG. 3) positioned within the body 101 of the tip assembly 100 via a respective duct 22, 24, 26 of the adapter 20 to create one or more independent chamber paths extending from the pipette controller 10 to the tip assembly 100. In embodiments the diameter of the ducts 22, 24, 26 of the adapter 20 may be smaller than the diameter of each respective coupled shaft in the pipette controller 10. The adapter 20 may direct or funnel the ducts 22, 24, 26 to correspond to the spatial arrangement of the chambers 104, 106, 108 (FIG. 3) within the tip assembly 100. In one embodiment, the pipette controller 10 includes three three-dimensionally spaced shafts within the shell 12 of the pipette controller 10 that are funneled by the adapter 20 via a first duct 22 to a first side chamber 106, via a second duct 26 to a second side chamber 108, and via a third duct 24 to an intake chamber 104 to correspond to the linear arrangement of the chambers 104, 106, 108 (FIG. 3) within the body 101 of the tip assembly 100.

The adapter 20 may contain a first side piston 116 at least partially positioned within the first duct 22, a second side piston 118 at least partially positioned within the second duct 26, and an intake piston 114 at least partially positioned within the third duct 24. The pistons 114, 116, 118 may be driven by the operation of the actuators within the shell 12 of the pipette controller 10. The independent chamber paths allow for each actuator of the pipette controller 10 to control a respective piston 114, 116, 118 independent of the other pistons 114, 116, 118. In embodiments, each piston 114, 116, 118 may be driven pneumatically by actuation of each linear rider to direct the movement of each piston 114, 116, 118 using compressed air. In some embodiments, each piston 114, 116, 118 may be directly coupled to each actuator to enable each actuator to control the positioning of each piston 114, 116, 118 through a direct mechanical coupling.

In embodiments, the pistons 114, 116, 118 are at least partially positioned within the tip assembly 100 such that the first side piston 116 is partially positioned within the first side chamber 106 (FIG. 3), the second side piston 118 is partially positioned within the second side chamber 108 (FIG. 3), and the intake piston 114 is partially positioned within the intake chamber 104 (FIG. 3). In some embodiments, the pistons 114, 116, 118 are coupled with the adapter 20 and can selectively enter the intake chamber 104, the first side chamber 106, and the second side chamber 108, respectively, via openings positioned at a first end 102 the tip assembly 100. The pistons 114, 116, 118 may extend into the intake chamber 104, first side chamber 106, and the second side chamber 108 respectively to directly contact the contents within the tip assembly 100. After use, the tip assembly 100 can be selectively removed from the adapter 20 and discarded, leaving the pistons 114, 116, 118 attached to the adapter 20. The pistons 114, 116, 118 can be cleaned and reused with a subsequent tip assembly 100. In some embodiments, the pistons 114, 116, 118 are coupled to the tip assembly 100. In embodiments, the intake chamber 104, the first side chamber 106, and the second side chamber 108 are partially covered or sealed to enable movement of pistons 114, 116, 118 within the tip assembly 100 while maintaining a secure seal at the first end 102 of the tip assembly 100. Each piston 114, 116, 118 may additionally include a top portion extending from the first end 102 to engage with each corresponding duct 22, 24, 26 within the adapter 20. The selective coupling of each piston 114, 116, 118 with each corresponding duct 22, 24, 26 places the pistons 114, 116, 118 in operative connection with each actuator within the shell 12 such that the pipette controller 10 can drive each piston 114, 116, 118 within the tip assembly 100. After use, the tip assembly 100 can be selectively removed from the adapter 20 and discarded with the pistons 114, 116, 118 still coupled to the tip assembly 100. Replacement pistons 114, 116, 118 may be provided with each subsequent tip assembly 100. This embodiment presents particular advantages because the pistons 114, 116, 118 would not need to be cleaned after each use.

Referring now to FIG. 3, the tip assembly 100 is depicted coupled to the adapter 20. The tip assembly 100 includes a body 101 (FIG. 2) forming an outer surface of the tip assembly 100. Furthermore, the body 101 defines the plurality of chambers 104, 106, 108 therein, extending from the first end 102 towards a second end 103. The first end 102 of the tip assembly 100 is positioned at an uppermost surface of the tip assembly 100 along a vertical axis. In embodiments, the first end 102 can selectively couple with the adapter 20 to position the intake chamber 104 proximate the third duct 24, position the first side chamber 106 proximate the first duct 22, and position the second side chamber 108 proximate the second duct 26. In embodiments, each chamber 104, 106, 108 is columnar with a smooth interior wall. In some embodiments, each chamber 104, 106, 108 includes features positioned on the interior walls like ridges, dimples, splines, or threads that can guide the movement of the pistons 114, 116, 118 through the chambers 104, 106, 108. In some embodiments, each chamber 104, 106, 108 has a circular cross-section, however, it should be understood each chamber 104, 106, 108 can have any suitable cross-section.

The tip assembly 100 includes the intake chamber 104 extending from the first end 102 to the second end 103. The tip assembly 100 may further include an inlet 120 that is in fluid communication with the intake chamber 104. In the embodiment depicted in FIG. 3, the inlet 120 extends beyond the second end 103, however, it should be understood that this is merely an example. In some embodiments, the inlet 120 may terminate at the second end 103. The inlet 120 may have a cone-shaped, tapered profile that can facilitate interaction with a biological sample. Further, the inlet 120 defines an opening along a bottommost portion of the inlet 120 for passing a biological sample into or out of the intake chamber 104. The intake chamber 104 includes the intake piston 114 positioned at least partially within the intake chamber 104 proximate the first end 102. In embodiments, the intake chamber 104 includes a mixer 110 positioned at least partially within the intake chamber 104 to interact with and process a biological sample. In some embodiments, the mixer 110 may rotate within the intake chamber 104 to chop, stir, and otherwise mix solid and/or liquid biological samples positioned within the intake chamber 104. In some embodiments, the mixer 110 may be partially positioned within the inlet 120 to interact with the biological sample before the biological sample enters the intake chamber 104. The mixer 110 may be mechanically coupled to the intake piston 114 such that the intake piston 114 can impart a rotational force on the mixer 110 through a direct coupling. In other embodiments, the intake piston 114 may impart a rotational force on the mixer 110 without direct contact via pneumatic means. In some embodiments, the rotation of the mixer 110 as the mixer 110 contacts a biological sample enables the helical threads or blades of the mixer 110 to draw or lift a portion of the biological sample into the intake chamber 104 through the inlet 120. In other embodiments, a user may directly insert a biological sample into the intake chamber 104 using a swab, syringe, or other comparable mechanism.

In some embodiments, the mixer 110 comprises stationary elements within the intake chamber 104. For example, in some embodiments, the mixer 110 comprises one or more louvers, baffles, or the like. In the embodiment depicted in FIG. 3, the mixer 110 comprises helically arranged baffles. As fluid is drawn into the intake chamber 104, either from outside of the tip assembly 100, from the first side chamber 106, or from the second side chamber 108, the louvers or baffles of the mixer 110 engage the biological sample causing the fluid to mix. In embodiments, fluids from the first side chamber 106 and/or the second side chamber 108 can be mixed with each other via the mixer 110 in the intake chamber 104 and/or with biological sample drawn in to the intake chamber 104.

The first side chamber 106 of the tip assembly 100 extends from the first end 102 towards the second end 103. The first side chamber 106 includes the first side piston 116 positioned at least partially within the first side chamber 106 proximate the first end 102. Additionally, the tip assembly 100 also includes the second side chamber 108 extending from the first end 102 toward the second end 103. The second side chamber 108 includes the second side piston 118 positioned at least partially within the second side chamber 108 proximate the first end 102. The first side chamber 106 and the second side chamber 108 may be positioned adjacent to the intake chamber 104 and may be placed in fluid communication with the intake chamber 104 to permit the passage of a fluid from both the first side chamber 106 and the second side chamber 108 into the intake chamber 104 to mix with a biological sample. In embodiments, the first side chamber 106, the second side chamber 108, and the intake chamber 104 are defined within the body 101 of the tip assembly 100 in a linear arrangement with the first side chamber 106 opposite the second side chamber 108, on either side of the intake chamber 104. In embodiments, the intake chamber 104, the first side chamber 106, and the second side chamber 108 have similar internal dimensions. In some embodiments, the intake chamber 104 has a first diameter and a first internal volume, while the first side chamber 106 and the second side chamber 108 have a second diameter and a second internal volume that are smaller than the first diameter and the first internal volume.

In embodiments, the tip assembly 100 includes at least one primary channel 122 extending between the first side chamber 106 and the intake chamber 104 to enable fluid communication between the first side chamber 106 and the intake chamber 104. The primary channel 122 is positioned proximate the second end 103 so that as the first side piston 116 is depressed, it creates a pressure gradient directing the contents of the first side chamber 106 from the first end 102 to the second end 103 and through the primary channel 122 into the intake chamber 104. Likewise, the tip assembly 100 further includes at least one secondary channel 124 extending between the second side chamber 108 and the intake chamber 104 to enable fluid communication between the second side chamber 108 and the intake chamber 104. The secondary channel 124 is positioned proximate the second end 103 so that as the second side piston 118 is depressed, it creates a pressure gradient directing the contents of the second side chamber 108 from the first end 102 towards the second end 103 and through the secondary channel 124 into the intake chamber 104. In embodiments, each of the primary channel 122 and the secondary channel 124 further includes a filter 152, 154 positioned to interact with a fluid or biological sample passing through the primary channel 122 or the secondary channel 124 to restrict the movement of large particles or other specific items of interest. In some embodiments, the filter 152, 154 defines openings sized to separate ova and/or parasites positioned from the remainder of the biological sample. In some embodiments, the filters 152, 154 may include multiple stages. For example, in some embodiments, the filters 152, 154 may include a first stage defining openings configured to allow ova and parasites to pass through the first stage, while restricting debris from passing through the first stage. A second stage of the filters 152, 154 may have openings configured to restrict the passage of ova and/or parasites from passing through, such that ova and parasites are captured at the second stage. Furthermore, one or both of the primary channel 122 and the secondary channel 124 may include a one-way valve 156, 158 to enable fluid to flow from the first side chamber 106 to the intake chamber 104 through the primary channel 122 or from the second side chamber 108 to the intake chamber 104 through the secondary channel 124. However, the first one-way valve 156 positioned in the primary channel 122 restricts fluid flow from the intake chamber 104 to the first side chamber 106 and the second one-way valve 158 positioned in the secondary channel 124 restricts fluid flow from the intake chamber 104 to the second side chamber 108.

In embodiments, the tip assembly 100 may include a first fluid medium retained within the first side chamber 106. The first fluid medium may come pre-loaded in the tip assembly 100 or the first fluid medium may be added to the tip assembly 100 by a user before adding a biological sample. The tip assembly 100 may also include a second fluid medium retained within the second side chamber 108. The second fluid medium may come pre-loaded in the tip assembly 100 or the second fluid medium may be added to the tip assembly 100 by a user before adding a biological sample. The first side chamber 106 may include a first pressure release seal 126 to retain and segregate the first fluid medium from the primary channel 122 until the first side piston 116 is actuated to break the first pressure release seal 126 and release the first fluid medium. Likewise, the second side chamber 108 may include a second pressure release seal 128 to retain and segregate the second fluid medium from the secondary channel 124 until the second side piston 118 is actuated to break the second pressure release seal 128 and release the first fluid medium. In embodiments, the first pressure release seal 126 and the second pressure release seal 128 extend across the cross-sections of the first side chamber 106 and the second side chamber 108 respectively to prevent the first fluid medium and the second fluid medium from interacting with the second end 103 until either the first side piston 116 or the second side piston 118 is actuated. In other embodiments, the first pressure release seal 126 is positioned along the interior wall of the first side chamber 106 to cover the opening to the primary channel 122 until the first side piston 116 is actuated. Likewise, the second pressure release seal 128 is positioned along the interior wall of the second side chamber 108 to cover the opening to the secondary channel 124 until the second side piston 118 is actuated. In embodiments, one or more pressure release seals 126, 128 may be positioned in the chambers 104, 106, 108 proximate the first end 102 to contain a fluid therein and/or to seal the chambers 104, 106, 108 prior to the coupling of the tip assembly 100 with the adapter 20 (FIG. 2).

In embodiments, one or more of the first fluid medium and the second fluid medium may be a diagnostic reagent used to react with the biological sample to indicate a chemical property of the biological sample. Furthermore, in embodiments, one or more of the first fluid medium and the second fluid medium may be a dye or staining fluid to mark or color at least a portion of the biological sample. In some embodiments, one or more of the first fluid medium and the second fluid medium may be a diluent to dilute a biological sample for testing. In some embodiments, the first fluid and/or the second fluid medium may preserve the biological sample. In non-limiting embodiments, the diluent may be any one of water, salt/water mixture, sugar/water mixture, acetone, acetonitrile, butanone, chlorobenzene chloroform, dichloromethane, dimethyl formamide, DMSO, ethanol, glycerol, hexane, isopropanol, methanol, polyethylene glycol (PEG-400), propylene glycol, solketal, toluene, xylene or any other comparable diluent that can dilute and/or preserve a biological sample. Additionally,

Still referring to FIG. 3, the tip assembly 100 may operate according to the following methods. The tip assembly 100 is selectively coupled to the pipette controller 10 (FIG. 2). As described above, the pipette controller 10 may include at least three independent actuators, such that a first actuator operatively drives the intake piston 114, a second actuator operatively drives the first side piston 116, and a third actuator operatively drives the second side piston 118. A biological sample can be loaded into the intake chamber 104 through the inlet 120. The biological sample can be introduced through a manual insertion method such as a swab, syringe, or other external device or the biological sample may be introduced to the intake chamber 104 due to interaction with the mixer 110. In embodiments in which the biological sample is a fluid, the biological sample can be introduced into the intake chamber 104 via pressure differential introduced via the intake piston 114.

Once the biological sample has been introduced to the intake chamber 104, the first side piston 116, which begins in a retracted position proximate the first end 102, can be moved or depressed toward the second end 103 to increase a first local pressure in the first side chamber 106. The increase in pressure within the first side chamber 106 forms a pressure gradient that directs the first fluid medium within the first side chamber 106 from the first end 102 toward the second end 103. As the first fluid medium moves toward the second end 103, it is directed through the primary channel 122 into the intake chamber 104. In embodiments, as the first side piston 116 is moved, the increase in local pressure within the first side chamber 106 breaks the pressure release seal 126 and enables the first fluid medium to flow through the primary channel 122 from the first side chamber 106 to the intake chamber 104 to mix with the biological sample.

The second side piston 118, which also begins in a retracted position proximate the first end 102, can be moved or depressed toward the second end 103 to increase a first local pressure in the second side chamber 108. The increase in pressure within the second side chamber 108 forms a pressure gradient that directs the second fluid medium within the second side chamber 108 from the first end 102 toward the second end 103. As the second fluid medium flows toward the second end 103, it is directed through the secondary channel 124 into the intake chamber 104. As the second side piston 118 is moved, the increase in local pressure within the second side chamber 108 breaks the pressure release seal 128 and enables the second fluid medium to flow through the secondary channel 124 from the second side chamber 108 to the intake chamber 104 to mix with the biological sample.

In some embodiments, the first side piston 116 and the second side piston 118 can be moved from the first end 102 towards the second end 103 simultaneously to introduce the first fluid medium and the second fluid medium into the intake chamber 104 at the same time to create turbulent flow and to more efficiently homogenize the mixture as compared to the introduction of the first and/or second fluid medium without turbulent flow. In some embodiments, the first side piston 116 and the second side piston 118 may be sequentially depressed to introduce the first fluid medium and the second fluid medium into the intake chamber 104 at different times. In embodiments, first fluid medium and the second fluid medium may have similar volumes, while in other embodiments they may have different volumes. In embodiments, the first side chamber 106 may not include a first fluid medium. In other embodiments, the second side chamber 108 may not include a second fluid medium. Once the biological sample, the first fluid medium, and the second fluid medium have sufficiently interacted within the intake chamber 104, the intake piston 114 can be moved or depressed from the first end 102 toward the second end 103 to increase the local pressure in the intake chamber 104 to direct the mixture to exit the intake chamber 104 through the inlet 120.

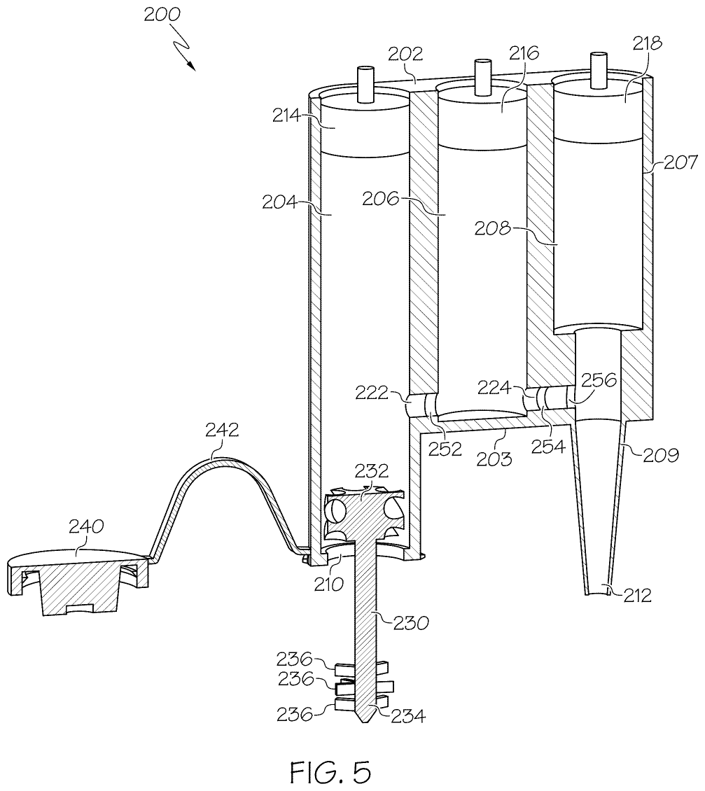

Referring now to FIGS. 4 and 5, another embodiment of a tip assembly 200 is depicted. The tip assembly 200 includes a body 201 forming an outer surface of the tip assembly 200. Furthermore, the body 201 defines an intake chamber 204, a mixing chamber 206, and an output chamber 208 therein, extending from a first end 202 of the body 201 towards a second end 203 of the body 201. The first end 202 of the body 201 is positioned above the second end 103 along a vertical axis. In embodiments, the first end 202 can selectively couple with the pipette controller 10 (FIG. 2) via the adapter 20 (FIG. 2) to position the intake chamber 204 proximate the first duct 22, position the mixing chamber 206 proximate the third duct 24, and position the output chamber 208 proximate the second duct 26. In embodiments, each chamber 204, 206, 208 is columnar with a smooth interior wall. In other embodiments, each chamber 204, 206, 208 is columnar and includes features positioned on the interior walls like ridges, dimples, splines, or threads that can guide the movement of one or more pistons 214, 216, 218 through the chambers 204, 206, 208. In some embodiments, each chamber 204, 206, 208 has a circular cross-sectional area, however, it should be understood each chamber 204, 206, 208 can have any suitable cross-sectional area.

Referring still to FIGS. 4 and 5, the intake chamber 204 extends from the first end 202 towards the second end 203. An inlet 210 is in fluid communication with the intake chamber 204. In embodiments, the inlet 210 is a circular aperture that permits access to the intake chamber 204, while in other embodiments the inlet 210 has a cylindrical or cone-shaped profile with an opening along a bottommost portion of the inlet 210 for passing a biological sample into or out of the intake chamber 204. The intake chamber 204 includes an intake piston 214 positioned at least partially within the intake chamber 204 proximate the first end 202. Furthermore, the intake chamber 204 includes a sample collection member 230 positioned at least partially within the intake chamber 204 to interact with and process the biological sample. In embodiments, the sample collection member 230 a hub 232 positioned within the intake chamber 204 and a head 234 extending from the hub 232 that can selectively enter or exit the intake chamber 204 through the inlet 210 to interact with the biological sample. The sample collection member 230 may be mechanically coupled to the intake piston 214 such that the intake piston 214 can move the position of the sample collection member 230 within the intake chamber 204. In some embodiments, the intake piston 214 may move the sample collection member 230 via pneumatic means. In some embodiments, the sample collection member 230 may extend through the inlet 210 such that the head 234 can engage the biological sample and then can be retracted (via the intake piston 214) to draw the head 234 back into the intake chamber 204 to position a portion of the biological sample within the intake chamber 204. In some embodiments, a user may directly insert a biological sample into the intake chamber 204 using a swab, syringe, or other comparable mechanism. In embodiments, the tip assembly 200 further includes a cap 240 having a corresponding size to the inlet 210. The cap 240 may be tethered to the body 201 by a flexible piece 242 and may be positioned within the inlet 210 to selectively engage the inlet 210 to selectively seal the intake chamber 204.

In embodiments, the sample collection member 230 may be a fecal collection tip. The hub 232 may include a plurality of groves, splines, threads, or other equivalent features that contact the interior wall of the intake chamber 204 and impart a rotational motion on the sample collection member 230 as it moves from the first end 202 towards the second end 203 or as the sample collection member 230 moves from the second end 203 to the first end 202. The sample collection member 230 also includes the head 234. In embodiments, the head 234 includes a plurality of bristles 236 extending radially from the head 234 to interact with and retain portions of the biological sample (e.g., a fecal sample). In embodiments, the head 234 and the plurality of bristles 236 rotate in conjunction with the hub 232 to chop, stir, and otherwise mix solid and/or liquid biological samples positioned outside or within the intake chamber 104.

The output chamber 208 extends from the first end 202 to the second end 203. The output chamber 208 includes an output piston 218 positioned at least partially within the output chamber 208 proximate the first end 202. An outlet 212 is in fluid communication with the output chamber 208 and may extend beyond the second end 203. In embodiments, the outlet 212 may have a cylindrical profile or a tapered dispensing tip with an opening along a bottommost portion of the outlet 212 for passing a biological sample out of the output chamber 208. Furthermore, the output chamber 208 may define multiple regions with disparate diameters. In embodiments, the output chamber 208 has two or more interior portions 207, 209 with different internal diameters. In embodiments, the first interior portion 207 proximate the first end 202 has a first diameter that is similar to the diameter of the intake chamber 204. The second interior portion 209 is in fluid communication with the first interior portion 207 and is proximate the second end 203 having a second diameter that is less than the first diameter.

The mixing chamber 206 extends from the first end 202 towards the second end 203. The mixing chamber 206 includes a mixing piston 216 positioned at least partially within the mixing chamber 206 proximate the first end 202. Unlike the intake chamber 204 and the output chamber 208, the mixing chamber 206 does not include an inlet or outlet, but instead forms a closed surface along the second end 203. In the embodiment depicted in FIGS. 4 and 5, the intake chamber 204, the mixing chamber 206, and the output chamber 208 are defined within the body 201 of the tip assembly 200 in a linear arrangement with the intake chamber 204 opposite the output chamber 208 on either side of the mixing chamber 206, however, it should be understood that this is merely an example. In embodiments, the intake chamber 204, the mixing chamber 206, and the output chamber 208 have similar internal dimensions (e.g., volumes, cross-sectional shapes, etc.). In some embodiments, the intake chamber 204, the mixing chamber 206, and the output chamber 208 each have different internal dimensions (e.g., volumes, cross-sectional shapes, etc.).

In embodiments, the tip assembly 200 includes one or more primary channels 222 extending between the intake chamber 204 and the mixing chamber 206 to enable fluid communication between the intake chamber 204 and the mixing chamber 206. Each primary channel 222 is positioned proximate the second end 103 so that as the intake piston 214 is depressed within the intake chamber 204, it creates a pressure gradient directing the contents of the intake chamber 204 from the first end 202 toward the second end 203 and through each primary channel 222 into the mixing chamber 206. By contrast, as the mixing piston 216 is depressed within the mixing chamber 206, it creates a pressure gradient directing the contents of the intake chamber 204 from the first end 202 toward the second end 203 and selectively through the primary channel 222. The tip assembly 200 further includes one or more secondary channels 224 extending between the mixing chamber 206 and the output chamber 208 to enable fluid communication between the mixing chamber 206 and the output chamber 208. Each secondary channel 224 is positioned proximate the second end 203 so that as the mixing piston 216 is depressed within the mixing chamber 206, it creates a pressure gradient directing the contents of the mixing chamber 206 from the first end 202 toward the second end 203 and through each secondary channel 224 into the output chamber 208. Additionally, as the output piston 218 is depressed within the output chamber 208, it creates a pressure gradient directing the contents of the output chamber 208 from the first end 202 to the second end 203 and through the outlet 212. Similarly, movement of the mixing piston 216 can cause contents of the mixing chamber 206 to move into the intake chamber 204 or through the secondary channel 224 into the output chamber 208, depending on the positioning of the intake piston 214 and the output piston 218.

In embodiments, each of the primary channel 222 and the secondary channel 224 includes a filter 252 positioned to interact with a fluid or biological sample passing through the primary channel 222 or the secondary channel 224 to restrict the movement of large particles or other specific items of interest. In some embodiments, the filter 252, 254 defines openings sized separate ova and/or eggs positioned within the biological sample from the remainder of the biological sample. In some embodiments, the filters 252, 254 may include multiple stages. For example, in some embodiments, the filters 252, 254 may include a first stage defining openings configured to allow ova and parasites to pass through the first stage, while restricting debris from passing through the first stage. A second stage of the filters 252, 254 may have openings configured to restrict the passage of ova and/or parasites from passing through, such that ova and parasites are captured at the second stage. Furthermore, the secondary channel 124 may include a one-way valve 256 to enable a fluid to flow from the mixing chamber 206 to the output chamber 208 through the secondary channel 224. However, the one-way valve 256 positioned in the secondary channel 224 restricts fluid flow from the output chamber 208 back into the mixing chamber 206.

In embodiments, the tip assembly 200 may include a fluid medium retained within the mixing chamber 206. The fluid medium may come pre-loaded in the tip assembly 200 or the first fluid medium may be added to the tip assembly 200 by a user before adding a biological sample. The mixing chamber 206 may include a pressure release seal 226 to retain and segregate the fluid medium from both the primary channel 222 and the secondary channel 224 until the mixing piston 216 is actuated to break the pressure release seal 226 and release the fluid medium. In embodiments, the pressure release seal 226 extends across the cross-section of the mixing chamber 206 to prevent the fluid medium from interacting with the second end 203 until the mixing piston 216 is actuated. In other embodiments, one or more pressure release seals 226 are positioned along the interior wall of the mixing chamber 206 to cover the opening to the primary channel 222 and/or the secondary channel 224 until the mixing piston 216 is actuated.

Still referring to FIGS. 4 and 5, the tip assembly 200 may operate according to the following methods. The tip assembly 200 may be selectively coupled to the pipette controller 10 (FIG. 2) such that the first actuator operatively drives the intake piston 214, the second actuator operatively drives the mixing piston 216, and the third actuator operatively drives the output piston 218. In embodiments, the intake piston 214 and the output piston 218 may begin in a depressed position, near to the second end 203 of the tip assembly 200. In embodiments, the mixing piston 216 begins in a withdrawn position proximate the first end 202. At least the head 234 of the sample collection member 230 may be positioned outside of the body 201 of the tip assembly 200 and brought into contact with a biological sample to retain a portion of the biological sample on the head 234. The first actuator may move the intake piston 214 to draw the sample collection member 230 and the collected biological sample into the intake chamber 204 through the inlet 210.

The second actuator moves the mixing piston 216 to increase a pressure in the mixing chamber 206 so that the fluid medium within the mixing chamber 206 is directed from the first end 202 to the second end 203 and through the primary channel 222 into the intake chamber 204 to form a mixture of the biological sample and the fluid medium. In embodiments, depressing the mixing piston 216 breaks the pressure release seal 226 to enable the fluid medium to flow through the primary channel 222 to the intake chamber 204. The second actuator then moves the mixing piston 216 to its withdrawn position and the first actuator moves the intake piston 214 to its depressed position to create a first pressure differential between the intake chamber 204 and the mixing chamber 206 so that the mixture is directed through the primary channel 222 into the mixing chamber 206. In some embodiments, the mixture interacts with a first filter 252 positioned within the primary channel 222 to separate ova, parasites, and other biological materials from the remainder of the biological sample. In some embodiments, for example in embodiments in which the biological sample is being prepared for antigen testing, the first filter 252 may assist in separating debris from the remainder of the biological sample. In embodiments, the first actuator and the second actuator cyclically alternate the positions of the intake piston 214 and the mixing piston 216 between the depressed and withdrawn positions to direct the mixture through the primary channel 222 to pass between the intake chamber 204 and the mixing chamber 206 two or more times to facilitate mixing.

The second actuator moves the mixing piston 216 to its depressed position and the third actuator moves the output piston 218 to its withdrawn position to create a second pressure differential between the mixing chamber 206 and the output chamber 208 so that the mixture is directed through the secondary channel 224 from the mixing chamber 206 into the output chamber 208. In embodiments, the mixture interacts with a second filter 254 positioned within the secondary channel 224 to separate ova, parasites, and other biological materials from the remainder of the biological sample. In embodiments, the secondary channel 224 includes a one-way valve 256 to permit the mixture to flow through the secondary channel 224 from the mixing chamber 206 to the output chamber 208, but the one-way valve 256 restricts flow of the mixture from the output chamber 208 back to the mixing chamber 206. Furthermore, the third actuator moves the output piston 218 to direct at least a portion of the mixture to exit the output chamber 208 through an outlet 212.

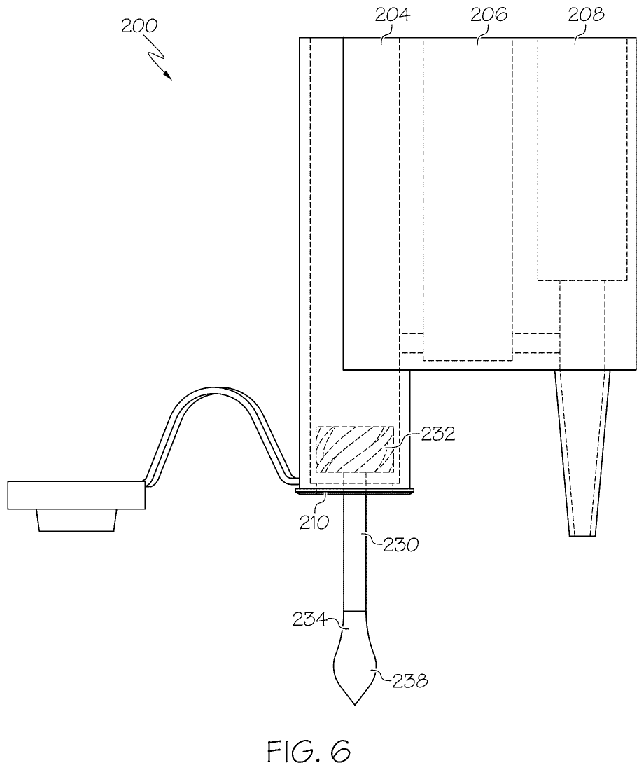

Referring now to FIG. 6, the tip assembly 200 is shown including another embodiment of a sample collection member 230A. In embodiments, the sample collection member 230A may be an ear collection tip. It should be appreciated that the sample collection member 230A is substantially similar to the sample collection member 230 illustrated in FIG. 5 and discussed herein. Accordingly, the sample collection member 230A includes the hub 232 that is retained within the intake chamber 204. However, the head 234 of the sample collection member 230A has a smooth, tapered point 238, rather than the bristles 236 of the sample collection member 230 (FIG. 5) to enable the sample collection member 230A to interface an animal's ear without causing discomfort or damage.

Referring now to FIG. 7, an illustrative embodiment of another tip assembly 300 is depicted. In the embodiment depicted in FIG. 7, the tip assembly 300 includes a testing chamber 308 for performing an assay, such as a lateral flow assay. The tip assembly 300 includes the body 301 defining the intake chamber 304, the mixing chamber 306, and the testing chamber 308 therein, extending from a first end 302 of the body 301 towards a second end 303 of the body 301. The first end 302 of the body 301 is positioned above the second end 303 of the body 301 along a vertical axis. In embodiments, the first end 302 can selectively couple with the pipette controller 10 (FIG. 2) via the adapter 20 (FIG. 2) to position the intake chamber 304 proximate the first duct 22, position the mixing chamber 306 proximate the third duct 24, and position the testing chamber 308 proximate the second duct 26. In embodiments, each chamber 304, 306, 308 is cylindrical with a smooth interior wall. In other embodiments, each chamber 304, 306, 308 includes features positioned on the interior walls like ridges, dimples, splines, or threads that can guide the movement of one or more pistons 314, 316, 318 through the chambers 304, 306, 308. In some embodiments, each chamber 304, 306, 308 has a circular cross-sectional area, however, it should be understood that in some embodiments each chamber 304, 306, 308 can have any suitably shaped cross-sectional.

The intake chamber 304 extends from the first end 302 towards the second end 303. An inlet 310 is in fluid communication with the intake chamber 304 and may extend beyond the second end 303. In embodiments, the inlet 310 is a circular aperture that permits access to the intake chamber 304, while in other embodiments the inlet 310 has a cylindrical or cone-shaped profile with an opening along a bottommost portion of the inlet 310 for passing a biological sample into or out of the intake chamber 304. The intake chamber 304 includes an intake piston 314 positioned at least partially within the intake chamber 304 proximate the first end 302. In some embodiments, the intake piston 314 can be moved to draw a fluid biological sample into the intake chamber 304. In other embodiments, a user may directly insert a biological sample into the intake chamber 304 using a swab, syringe, or other comparable mechanism.

The mixing chamber 306 extends from the first end 302 towards the second end 303. The mixing chamber 306 includes a mixing piston 316 positioned at least partially within the mixing chamber 306 proximate the first end 302. The mixing chamber 306 does not include an opening along the second end 303, but instead forms a closed surface along the second end 303. In embodiments, the mixing chamber 306 retains a fluid conjugate material. The fluid conjugate material may be initially segregated from interacting with the biological sample in the intake chamber 304 via one or more pressure release seals until the mixing piston 316 is actuated to release the conjugate. The conjugate may chemically interact with the biological sample, as will be understood by those of skill in the art.

The testing chamber 308 is configured for performing an assay, and extends from the first end 302 towards the second end 303. The testing chamber 308 includes a testing piston 318 positioned at least partially within the testing chamber 308 proximate the first end 302. The testing chamber 308 does not include an opening along the second end 303, but instead forms a closed surface along the second end 303. Furthermore, the testing chamber 308 includes a matrix 330 positioned proximate the second end 303, a first region 350 positioned proximate the first end 302 for containing a reagent, and a second region 340 containing a wash positioned between the first region 350 and the matrix 330 within the testing chamber 308. The first region 340 may be segregated from the matrix 330 by a first pressure release tab 334 extending across the inner diameter of the testing chamber 308. Additionally, the second region 350 may be segregated from the first region 340 by a second pressure release tab 336 extending across the inner diameter of the testing chamber 308. In embodiments, the testing chamber 308 may also include an expansion field 332 positioned between the matrix 330 and the first pressure release tab 334. The expansion field 332 may be a portion of the testing chamber 308 having a larger diameter to facilitate the mixing of the reagent with the biological sample at or near the matrix 330.

In embodiments, the intake chamber 304, the mixing chamber 306, and the testing chamber 308 are defined within the body 301 of the tip assembly 300 in a linear arrangement with the intake chamber 304 opposite the testing chamber 308 on either side of the mixing chamber 306. In embodiments, each of the intake chamber 304, the mixing chamber 306, and the testing chamber 308 have similar internal dimensions. In some embodiments, the intake chamber 304, the mixing chamber 306, and the testing chamber 308 each have different internal dimensions/volumes.

The tip assembly 300, in embodiments, includes one or more primary channels 322 extending between the intake chamber 304 and the mixing chamber 306 to enable fluid communication between the intake chamber 304 and the mixing chamber 306. Each primary channel 322 is positioned proximate the second end 303 so that as the intake piston 314 is depressed within the intake chamber 304, it creates a pressure gradient directing the contents of the intake chamber 304 from the first end 302 to the second end 303 and through each primary channel 322 into the mixing chamber 306. In some embodiments, as the mixing piston 316 is depressed within the mixing chamber 306, it creates a pressure gradient directing the contents of the mixing chamber 306 from the first end 302 to the second end 303 and selectively through either one of the primary channels 322 into the intake chamber 304 or through a secondary channel 324 between the mixing chamber 306 and the testing chamber 308, depending on the positioning of the intake piston 314 and the testing piston 318. The one or more secondary channels 324 are positioned proximate the second end 303 so that as the mixing piston 316 is depressed within the mixing chamber 306, it creates a pressure gradient directing the contents of the mixing chamber 306 from the first end 302 to the second end 303 and through each secondary channel 324 into the testing chamber 308. Additionally, as the testing piston 318 is depressed within the testing chamber 308, it creates a pressure gradient that breaks the first pressure release tab 334 and the second pressure release tab 336 and directs the reagent and the wash to flow from towards the second end 303 to interact with the matrix 330.

In some embodiments, each of the primary channels 322 and the secondary channels 324 further includes a filter 352, 354 positioned to interact with a fluid or biological sample passing through the primary channel 322 or the secondary channel 324 to restrict the movement of large particles or other specific items of interest. In some embodiments, the filter 352, 354 defines openings sized to separate ova and/or eggs positioned within the biological sample from the remainder of the biological sample. Furthermore, the secondary channel 324 may include a one-way valve 356 to enable fluid to flow from the mixing chamber 306 to the testing chamber 308 through the secondary channel 324. However, the one-way valve 356 positioned in the secondary channel 324 restricts fluid flow from the testing chamber 308 back into the mixing chamber 306.

The tip assembly 300 may generally function according to the method described above for use with the tip assembly 200. The tip assembly 300 has one operational difference in that the mixture including the biological sample, the wash, the reagent, and optionally a conjugate are not dispensed from the testing chamber 308. Instead, the mixture interacts with the matrix 330 to visually indicate a test result depending on the interaction of the biological sample, the reagent, and the matrix 330.

Accordingly, embodiments of the present disclosure provide for a multi-chambered tip assembly to facilitate rapid, cost-effective sample preparation of biological samples. The systems and methods described herein provide improved capabilities compared to traditional pipette assemblies. The present disclosure provides for systems and methods that can eliminate the need for intermediate preparation steps. Additionally, the single consumable tips described herein eliminate the need for purchasing multiple consumable products, thereby reducing preparation costs. Furthermore, the automated and pre-measured sample preparation methods discussed herein will increase the precision and repeatability of sample preparation steps. The independent, tip assembly design provides for a sample preparation platform that can not only dispense prepared samples, but also mix, aerate, dilute, filter, separate, and test biological samples within a single consumable apparatus.

It may be noted that one or more of the following claims utilize the terms “where,” “wherein,” or “in which” as transitional phrases. For the purposes of defining the present technology, it may be noted that these terms are introduced in the claims as an open-ended transitional phrase that are used to introduce a recitation of a series of characteristics of the structure and should be interpreted in like manner as the more commonly used open-ended preamble term “comprising.”

Having described the subject matter of the present disclosure in detail and by reference to specific embodiments, it may be noted that the various details described in this disclosure should not be taken to imply that these details relate to elements that are essential components of the various embodiments described in this disclosure, even in casings where a particular element may be illustrated in each of the drawings that accompany the present description. Rather, the claims appended hereto should be taken as the sole representation of the breadth of the present disclosure and the corresponding scope of the various embodiments described in this disclosure. Further, it will be apparent that modifications and variations are possible without departing from the scope of the appended claims.

Further aspects of the embodiments herein are provided by the subject matter of the following clauses:

A tip assembly comprising: a body including a first end, a second end opposite the first end and defining a plurality of chambers therein, the plurality of chambers comprising: an intake chamber defining an inlet for passing a biological sample and comprising an intake piston positioned at least partially in the intake chamber; a mixer positioned at least partially within the intake chamber; a first side chamber positioned in communication with the intake chamber and comprising a first side piston positioned at least partially within the first side chamber; a second side chamber in communication with the intake chamber and comprising a second side piston positioned at least partially within the second side chamber a primary channel extending between the first side chamber and the intake chamber; and a secondary channel extending between the second side chamber and the intake chamber.

The tip assembly of any preceding clause, wherein the mixer extends from the first end to a position beyond the second end and comprises helical threads.

The tip assembly of any preceding clause, wherein the mixer is coupled to the intake piston and the helical threads cause the mixer to rotate as the mixer moves along the intake chamber.

The tip assembly of any preceding clause, further comprising a first fluid medium within the first side chamber.

The tip assembly of any preceding clause, the first side chamber further comprising a pressure release seal to retain and segregate the first fluid medium from the primary channel until the first side piston is actuated to break the pressure release seal and release the first fluid medium.

The tip assembly of any preceding clause, further comprising a second fluid medium within the second side chamber.

The tip assembly of any preceding clause, the second side chamber further comprising a pressure release seal to retain and segregate the second fluid medium from the secondary channel until the second side piston is actuated to break the pressure release seal and release the second fluid medium.

The tip assembly of any preceding clause, wherein each of the primary channel and the secondary channel further each comprises a filter positioned to interact with the biological sample passing through the primary channel or the secondary channel.

The tip assembly of any preceding clause, wherein each of the primary channel and the secondary channel further comprises a one-way valve to restrict a fluid flow out of the intake chamber.

The tip assembly of any preceding clause, the intake chamber having a first diameter and both the first side chamber and the second side chamber having a second diameter that is less than the first diameter.

A method for using a tip assembly, the method comprising: loading a biological sample into an intake chamber through an inlet; moving an intake piston to rotate a mixer operatively coupled to the intake piston within the intake chamber; moving a first side piston to increase a first local pressure in a first side chamber such that a first fluid medium contained within the first side chamber is directed through a primary channel into the intake chamber to form a mixture of the biological sample and the first fluid medium; moving a second side piston to increase a second local pressure in a second side chamber such that a second fluid medium contained within the second side chamber is directed through a secondary channel into the intake chamber to add the second fluid medium to the mixture; and moving the intake piston to direct the mixture to exit the intake chamber through the inlet.

The method of any preceding clause, further comprising selectively coupling the tip assembly with a pipette controller.

The method of any preceding clause, the pipette controller comprising at least three independent actuators, wherein a first actuator operatively drives the intake piston, a second actuator operatively drives the first side piston, and a third actuator operatively drives the second side piston.

The method of any preceding clause, further comprising simultaneously depressing the first side piston and the second side piston.

The method of any preceding clause, further comprising sequentially depressing the first side piston and the second side piston.

The method of any preceding clause, the first fluid medium comprising a solvent or diagnostic reagent.

The method of any preceding clause, the second fluid medium comprising a dye or staining fluid.

The method of any preceding clause, further comprising rotating the mixer to introduce the biological sample into the intake chamber.

The method of any preceding clause, providing a pressure release seal within the first side chamber.

The method of any preceding clause, providing a pressure release seal within the second side chamber.

The method of any preceding clause, wherein each of the primary channel and the secondary channel further comprises a one-way valve to inhibit flow of the first fluid medium and the second fluid medium out of the intake chamber.

The method of any preceding clause, the intake chamber having a first diameter and both the first side chamber and the second side chamber having a second diameter that is less than the first diameter.

A tip assembly comprising: a body defining a first end, a second end opposite the first end, and a plurality of chambers therein, the plurality of chambers comprising: an intake chamber defining an inlet at the second end for passing a biological sample and comprising an intake piston positioned at least partially in the intake chamber; an output chamber defining an outlet at the second end for passing the biological sample and comprising an output piston positioned at least partially in the output chamber; a mixing chamber positioned between the intake chamber and the output chamber and comprising a mixing piston positioned at least partially in the mixing chamber; a primary channel extending between the intake chamber and the mixing chamber; and a secondary channel extending between the mixing chamber and the output chamber.

The tip assembly of any preceding clause, further comprising a sample collection member at least partially positioned within the intake chamber and movable within the intake chamber through the inlet.

The tip assembly of any preceding clause, the sample collection member comprising a fecal collection tip, the fecal collection tip further comprising a head and a hub opposite the head, wherein the hub is positioned within the intake chamber and the head further comprises a plurality of bristles extending radially from the head to engage the biological sample.

The tip assembly of any preceding clause, the sample collection member comprising an ear collection tip, the ear collection tip further comprising a head and a hub opposite the head, wherein the hub is positioned within the intake chamber and the head further comprises a tapered point to engage the biological sample.

The tip assembly of any preceding clause, wherein the second end of the body is selectively coupled with a pipette controller.

The tip assembly of any preceding clause, further comprising a fluid medium provided within the mixing chamber.

The tip assembly of any preceding clause, the mixing chamber further comprising a pressure release seal to retain and segregate the fluid medium from the primary channel and the secondary channel until the mixing piston is actuated to break the pressure release seal and release the fluid medium.

The tip assembly of any preceding clause, further comprising a first filter positioned within the primary channel.

The tip assembly of any preceding clause, further comprising a second filter positioned within the secondary channel.

The tip assembly of any preceding clause, the intake chamber further comprising a cap having a corresponding size to engage the inlet to selectively seal the intake chamber.

The tip assembly of any preceding clause, the outlet further comprising a tapered dispensing tip extending from the first end.

The tip assembly of any preceding clause, wherein the output chamber includes a first interior portion at the second end having a first diameter and a second interior portion at the first end having a second diameter less than the first diameter.

A method for using a tip assembly, the method comprising: moving an intake piston to draw a sample collection member and a collected biological sample into an intake chamber; moving a mixing piston to increase a pressure in a mixing chamber such that a fluid medium within the mixing chamber is directed through a primary channel into the intake chamber to form a mixture of the biological sample and the fluid medium; moving the mixing piston and moving the intake piston to create a first pressure differential between the intake chamber and the mixing chamber such that the mixture is directed through the primary channel into the mixing chamber; moving an output piston and moving the mixing piston to create a second pressure differential between the mixing chamber and an output chamber such that the mixture is directed through a secondary channel into the output chamber; and moving the output piston to direct at least a portion of the mixture to exit the output chamber through an outlet.

The method of any preceding clause, further comprising selectively coupling the tip assembly with a pipette controller.

The method of any preceding clause, the pipette controller comprising three independent actuators, wherein a first actuator operatively drives the intake piston, a second actuator operatively drives the mixing piston, and a third actuator operatively drives the output piston.

The method of any preceding clause, further comprising cyclically directing the mixture between the intake chamber and the mixing chamber through the primary channel to facilitate mixing.

The method of any preceding clause, further comprising cyclically directing the mixture between the mixing chamber and the output chamber through the secondary channel to facilitate mixing.

The method of any preceding clause, further comprising filtering the mixture using a first filter positioned within the primary channel.

The method of any preceding clause, further comprising filtering the mixture using a second filter positioned within the secondary channel.

A tip assembly comprising: a pipette controller comprising a first actuator and a second actuator; a tip assembly selectively coupled to the pipette controller, the tip assembly comprising: an intake chamber defining an inlet for passing a biological sample; an intake piston positioned at least partially within the intake chamber and coupled to the first actuator; a sample collection member at least partially positioned within the intake chamber and movable within the intake chamber through the inlet to collect the biological sample and transport the biological sample into the intake chamber; an output chamber defining an outlet for passing the biological sample; an output piston positioned at least partially within the output chamber and coupled to the second actuator; and a channel extending between the intake chamber and the output chamber to enable fluid communication between the intake chamber and the output chamber.

The tip assembly of any preceding clause, the sample collection member comprising a fecal collection tip, the fecal collection tip further comprising a head and a hub opposite the head, wherein the hub is positioned within the intake chamber and the head further comprises a plurality of bristles extending radially from the head to engage the biological sample.

The tip assembly of any preceding clause, the sample collection member comprising an ear collection tip, the ear collection tip further comprising a head and a hub opposite the head, wherein the hub is positioned within the intake chamber and the head further comprises a tapered point to engage the biological sample.

The tip assembly of any preceding clause, wherein a fluid medium is provided within the output chamber.

The tip assembly of any preceding clause, wherein the output chamber comprises a pressure release seal to retain the fluid medium within the output chamber.

The tip assembly of any preceding clause, further comprising a filter positioned within the channel.

The tip assembly of any preceding clause, wherein the filter defines openings sized to separate ova and/or parasites positioned within the biological sample from a remainder of the biological sample.

The tip assembly of any preceding clause, further comprising a cap having a corresponding size to engage the inlet and selectively seal the intake chamber.

The tip assembly of any preceding clause, further comprising a tapered dispensing tip extending from the output chamber.

While particular embodiments have been illustrated and described herein, it should be understood that various other changes and modifications may be made without departing from the scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed:

1. A tip assembly comprising:
a body including a first end, a second end opposite the first end and defining a plurality of chambers therein, the plurality of chambers comprising:
an intake chamber defining an inlet for passing a biological sample and comprising an intake piston positioned at least partially in the intake chamber;
a mixer positioned at least partially within the intake chamber;
a first side chamber positioned in communication with the intake chamber and comprising a first side piston positioned at least partially within the first side chamber;
a second side chamber in communication with the intake chamber and comprising a second side piston positioned at least partially within the second side chamber
a primary channel extending between the first side chamber and the intake chamber; and
a secondary channel extending between the second side chamber and the intake chamber.

2. The tip assembly of claim 1, wherein the mixer extends from the first end to a position beyond the second end and comprises helical threads.

3. The tip assembly of claim 2, wherein the mixer is coupled to the intake piston and the helical threads cause the mixer to rotate as the mixer moves along the intake chamber.

4. The tip assembly of claim 1, further comprising a first fluid medium within the first side chamber.

5. The tip assembly of claim 4, the first side chamber further comprising a pressure release seal to retain and segregate the first fluid medium from the primary channel until the first side piston is actuated to break the pressure release seal and release the first fluid medium.

6. The tip assembly of claim 1, further comprising a second fluid medium within the second side chamber, wherein the second side chamber further comprises a pressure release seal to retain and segregate the second fluid medium from the secondary channel until the second side piston is actuated to break the pressure release seal and release the second fluid medium.

7. The tip assembly of claim 1, wherein each of the primary channel and the secondary channel further each comprises a filter positioned to interact with the biological sample passing through the primary channel or the secondary channel.

8. A tip assembly comprising:
a body defining a first end, a second end opposite the first end, and a plurality of chambers therein, the plurality of chambers comprising:
an intake chamber defining an inlet at the second end for passing a biological sample and comprising an intake piston positioned at least partially in the intake chamber;
an output chamber defining an outlet at the second end for passing the biological sample and comprising an output piston positioned at least partially in the output chamber;
a mixing chamber positioned between the intake chamber and the output chamber and comprising a mixing piston positioned at least partially in the mixing chamber;
a primary channel extending between the intake chamber and the mixing chamber; and
a secondary channel extending between the mixing chamber and the output chamber.

9. The tip assembly of claim 8, further comprising a sample collection member at least partially positioned within the intake chamber and movable within the intake chamber through the inlet.

10. The tip assembly of claim 9, the sample collection member comprising a fecal collection tip, the fecal collection tip further comprising a head and a hub opposite the head, wherein the hub is positioned within the intake chamber and the head further comprises a plurality of bristles extending radially from the head to engage the biological sample.

11. The tip assembly of claim 9, the sample collection member comprising an ear collection tip, the ear collection tip further comprising a head and a hub opposite the head, wherein the hub is positioned within the intake chamber and the head further comprises a tapered point to engage the biological sample.

12. The tip assembly of claim 8, further comprising a fluid medium provided within the mixing chamber, the mixing chamber further comprising a pressure release seal to retain and segregate the fluid medium from the primary channel and the secondary channel until the mixing piston is actuated to break the pressure release seal and release the fluid medium.

13. The tip assembly of claim 8, further comprising:
a first filter positioned within the primary channel; and
a second filter positioned within the secondary channel.

14. The tip assembly of claim 8, the outlet further comprising a tapered dispensing tip extending from the first end.

15. The tip assembly of claim 8, wherein the output chamber includes a first interior portion at the second end having a first diameter and a second interior portion at the first end having a second diameter less than the first diameter.

16. A tip assembly comprising:
a pipette controller comprising a first actuator and a second actuator;
a tip assembly selectively coupled to the pipette controller, the tip assembly comprising:
an intake chamber defining an inlet for passing a biological sample;
an intake piston positioned at least partially within the intake chamber and coupled to the first actuator;
a sample collection member at least partially positioned within the intake chamber and movable within the intake chamber through the inlet to collect the biological sample and transport the biological sample into the intake chamber;
an output chamber defining an outlet for passing the biological sample;
an output piston positioned at least partially within the output chamber and coupled to the second actuator; and
a channel extending between the intake chamber and the output chamber to enable fluid communication between the intake chamber and the output chamber.

17. The tip assembly of claim 16, the sample collection member comprising a fecal collection tip, the fecal collection tip further comprising a head and a hub opposite the head, wherein the hub is positioned within the intake chamber and the head further comprises a plurality of bristles extending radially from the head to engage the biological sample.

18. The tip assembly of claim 16, the sample collection member comprising an ear collection tip, the ear collection tip further comprising a head and a hub opposite the head, wherein the hub is positioned within the intake chamber and the head further comprises a tapered point to engage the biological sample.

19. The tip assembly of claim 16, wherein a fluid medium is provided within the output chamber, wherein the output chamber comprises a pressure release seal to retain the fluid medium within the output chamber.

20. The tip assembly of claim 16, further comprising a filter positioned within the channel, wherein the filter defines openings sized to separate ova and/or parasites positioned within the biological sample from a remainder of the biological sample.
